(19) United States
(12) Patent Application Publication
(10) Pub. No.: US 2010/0324689 A1
(43) Pub. Date: Dec. 23, 2010

(54) IMPLANT COMPRISING THERMOPLASTIC ELASTOMER

(76) Inventors: Darren Donald Obrigkeit, Aachen (DE); Atze Jan Nijenhuis, Sittard (NL)

Correspondence Address: NIXON & VANDERHYE, PC, 901 NORTH GLEBE ROAD, 11TH FLOOR, ARLINGTON, VA, 22203, US

(21) Appl. No.: 12/740,828

(22) PCT Filed: Oct. 30, 2008
(86) PCT No.: PCT/EP08/64756

§ 371 Date: Aug. 24, 2010

Related U.S. Application Data
(63) Continuation-in-part of application No. 61/000,904, filed on Oct. 30, 2007.

(30) Foreign Application Priority Data
Feb. 15, 2008 (EP) 08151530.6

(51) Int. Cl. A61F 2/44

(52) U.S. Cl. 623/17.16

(57) ABSTRACT

The invention relates to an artificial spinal implant comprising a thermoplastic elastomer comprising a hard phase and soft phase, wherein the hard phase comprises a polymer chosen from the group consisting of polyester, polyamide, polystyrene, polyacrylate and polyolefin and the soft phase comprises a polymer chosen from the group consisting of polyether, polyester, polyacrylate, polyolefin and polysiloxane.

The invention relates to an artificial spinal implant. The invention further relates to the use of a thermoplastic elastomer (TPE) in artificial spinal implants and in procedures for motion preservation in the spine.

Approximately one third to a quarter of the length of the adult human spine is occupied by the vertebral discs. Each disc comprises an annular wall (annular fibrosus) that surrounds and contains a central nucleus (nucleus pulposus) filled with gelatinous material that occupies approximately 30 to 50% of the cross sectional area of the disc. The annular wall is a concentrically laminated structure containing aligned collagen fibres and fibrocartilage and provides the major stabilizing structure to resist torsional and bending forces applied to the disc. The discs are contained between vertebral endplates comprised of hyaline cartilage that act as an intermediate layer between the hard vertebrae and the softer material of the disc.

The joints and muscoskeletal tissues of the human body are subject to traumatic injury and disease and degenerative processes that over a period of time can lead to the deterioration or failure of the joint causing severe pain or immobility. Generally, the ability of a joint to provide pain free articulation and carry load is dependent upon the presence of healthy bone, cartilage and associated musculoskeletal tissues that provide a stable joint. With reference to the spine, spinal disc degeneration, characterized by features such as loss of fluid, annular tears and myxomatous changes can result in discogenic pain and/or disc bulging or herniation of the nucleus in which the disc protrudes into the intervertebral foramen comprising spinal verves resulting in back pain and/pr sciatica. This condition is more commonly referred to as a “slipped” disc.

To alleviate the condition described above, the damaged spinal disc may be surgically removed from the spine and the two adjacent vertebrae either side of the damaged disc fused together (arthrodesis). Although this technique successfully eliminates the symptoms of pain and discomfort and improves joint stability, it results in a total loss of movement of the fused vertebral joint and increases the stress placed on the adjacent joints leading to collateral damage of these joints and associated soft tissues.

A more desired solution is to replace or repair the damaged spinal disc with an artificial implant that preserves pain free movement of the vertebrae and which mimics the motion and function of the healthy spine.

Among motion-preserving spinal implants the following classes can readily be identified:
1. Lumbar Total Disc Replacements—implants which replace and mimic the function of spinal discs in the lower spine.
2. Cervical Total Disc Replacements—implants which replace and mimic the function of spinal discs in the upper spine.
3. Interspinous Process Spacers—implants which increase or maintain the spacing between two vertebrae and limit the degree of extension and/or flexion in the spine. Typically, these implants are attached to the spinous vertebrae processes and/or nearby ligaments.
4. Dynamic Stabilization Implants—implants which increase or maintain the spacing between two vertebrae and “stabilize” them to maintain disc height and reduce the risk of spondylolisthesis (slipped discs). Typically these implants are attached via two pedicle screws placed through the left and right pedicles at each implanted vertebral level.
5. Nucleus Replacement Implants—implants which replace and mimic the nucleus pulposis (core) of the spinal disc.

Of these implant classes, disc replacements, also known as “artificial discs” have the most established clinical history. However, the development of existing artificial discs has been limited because they lack the complexity of structure and the materials they are composed of cannot adequately mimic the biomechanics of a normal healthy human spinal disc. Artificial disc replacements fall into two main categories:
1. conventional artificial discs, which act as an articulating joint, and
2. compliant artificial discs, which act as a damping junction between the vertebrae.

Conventional artificial discs articulate by using a bearing surface manufactured from metals, for example titanium and stainless steel, alloys or durable polymers including ultra-high molecular weight polyethylene (UHMWPE) and polyetherether ketones (PEEK). However, the use of hard, non-deformable bearing surfaces render the implant non-compliant and unable to replicate the compliant load bearing capacity provided by the natural disc. As a result, adjacent spinal levels are still exposed to increased mechanical stresses resulting in a high risk of further degeneration. In addition, wear particles are created by articulating implants; these particles are frequently the cause of complications such as inflammation.

Furthermore, conventional artificial discs struggle to reproduce the natural center of rotation in the spine. In many of such spinal implants two pivot points are applied (ball-and-socket designs, where pivot points exist 1.) in the disc space and 2.) at the facet joints) where one exists in nature, at the facet joints. This results in unnatural loading, pain, and degeneration of facet joints (EuroSpine 2007 conference proceedings, Posters 174, 178). Moreover, such discs are frequently expelled, which appears to be one of the primary complications of total disc replacement (FDA MAUDE database reports MW5003435, MW1035121; Spine. 28 (0) Journal of Spinal Disorders & Techniques: Special Online-Only Supplement to Spine:369-383, August 2003).

Examples of conventional artificial discs are given in Table 1.

TABLE 1
Conventional artifical lumbar disc replacement implants.

| Company | Implant | Reference |
|---|---|---|
| DePuy Spine | Charité | U.S. Pat. No. 5,401,269 |
| Disc Motion | TrueDisc PL | EP1738722A2 |
| Globus Medical | Alliance TDR | US2005/0043800A1 |
| LDR Spine | Mobidisc | US2006/0136063A1 |
| Medtronic Sofamor Danek | Maverick | US2007/0179615A1, |
| | | US2007/0179618A1 |
| SeaSpine/Alphatec | La Jolla | — |
| Spinal Kinetics | M6 | US7153325B2 |
| SpinalMotion | Kineflex | WO2006/014830A1 |
| Stryker Spine (SpineCore) | FlexiCore | US7258699B2 |
| Synthes | ProDisc | U.S. Pat. No. 6,936,071 |
| Vertebron | MDP | US2004/0172021A1 |
| Zimmer Spine | Dynardi | US2005/0261772A1 |

Compliant artificial spinal discs are generally either manufactured using a material of single uniform modulus (single durometer) or using two (dual durometer) or more materials of different modulus, in which case the material has a lower modulus core contained within a higher modulus shell. The former requires a compromise in material specification to balance strength and wear resistance with compliance. The latter often generates problems caused by a progressive failure along the interface between the two materials over a period of use. An artificial spinal disc of the latter type is known from U.S. Pat. No. 5,171,281.

Examples of compliant lumbar artificial disc replacement implants are given in Table 2.

TABLE 2
Compliant artifical lumbar disc replacement implants.

| Company | Implant | Device type | Reference |
|---|---|---|---|
| AxioMed Spine | Freedom | Elastomer | U.S. Pat. No. 7,169,181 |
| Biomet EBI | MIN T | Elastomer | — |
| GM Reis | Flexdisc | — | — |
| Nexgen Spine | Physio-L | Elastomer | US2007/0032874A1, |
| | | core/annulus | US2005/0015150A1 |
| Ranier Technology | CA Disc | One piece - | US2007/0043443A1, |
| | | polyurethane | US2007/0050038A1 |
| Spinal Kinetics | M6 | Elastomer | US2007/0050032A1 |
| Theken Disc | eDISC | Elastomer | US2006/0259143A1 |

In U.S. Patent Application 200710050038A1 an artificial implant is disclosed which comprises a body comprising at least a first and second polyurethane, the body having a pre-determined portion exhibiting a gradual variation in Young's modulus. A disadvantage of the above artificial implant is that polyurethane shows considerable creep upon stress, which may cause a change of shape of the artificial implant in time and corresponding loss of disc height. Moreover, polyurethanes show strain softening behaviour which also negatively affects relevant mechanical properties. Also, polyurethanes are known to degrade in aqueous environments.

Alternatively, silicone rubbers or combinations of silicone rubbers with other materials may be used in implants. However several complications are associated with silicone rubbers. For example, high performance silicone rubber is used in space-filler type joints in artificial joint replacement. One of the problems that occurs with these artificial replacements is that they can fail because the silicone rubber used for their fabrication is a relatively weak material and shown to break apart and segment (“Preparation and bioactivity of novel multiblock thermoplastic elastomer/tricalcium phosphate composites”, M. El Fray, Journal of Materials Science: Materials in Medicine, Volume 18, Number 3, March 2007, pp. 501-506 (6)). Other possible adverse effects of silicones used in implants are adsorption of oxidized lipids, which causes swelling and slight dimensional change, and insufficient chemical stability of siloxane bonds in specific physiological environments. Moreover, immunological reactions to silicone can also develop that can be local, regional due to silicone migration, or systemic. Migration of silicone has been documented on numerous occasions in the literature. Systemic reactions, such as acute renal insufficiency and respiratory compromise, etc., have been reported following the introduction of silicone into the body (Biomedical application of commercial polymers and novel polyisobutylene-based TPE for soft tissue replacement, J. E. Puskas, Biomacromolecules, Vol 5-4, July/August 2004).

A need therefore remains for an artificial spinal implant, for example an artificial spinal disc, which can be surgically inserted in place of the damaged spinal part and which will enable full, pain-free movement of the affected vertebral joint, which is durable enough to withstand the loads and wear imposed upon it in use without failing, and at the same time exhibit biomechanics which are as similar as possible to that of the body's own natural spinal parts and can so withstand both compression and torsional loading. If these requirements are not adequately met, and the artificial implant, for example disc, is too stiff, it will not deform sufficiently during movement and excessive deformation of the adjacent natural discs will occur. On the other hand, if the implant, for example disc, does not have the required degree of stiffness, excessive movement of the implant, for example disc, will occur causing it to bulge out resulting in pain and discomfort of the patient.

The aim of the invention is therefore to provide a material to be used in an artificial spinal implant, that provides an artificial spinal implant that does not show the aforementioned disadvantages, or at least shows them to a lesser extent.

This aim is achieved with an artificial spinal implant comprising a thermoplastic elastomer comprising a hard phase and soft phase, wherein the hard phase comprises a polymer chosen from the group consisting of polyester, polyamide, polystyrene, polyacrylate and polyolefin and the soft phase comprises a polymer chosen from the group consisting of polyether, polyester, polyacrylate, polyolefin and polysiloxane.

Surprisingly it has been found that the spinal implant according to the invention has superior shock-absorbing properties, flexibility, creep resistance, compression set and chemical resistance such that a compliant durable spinal implant can be made.

A spinal implant may comprise only one part. Alternatively, the implant may consist of two or more parts of which at least one part is made of the TPE according to the invention. As such, because of its superior adhesion properties the TPE can be combined with other elastomeric materials of different stiffness and flexibility and/or hard materials, such as metals and higher modulus polymers.

Another advantage of using the TPE according to the invention in a spinal implant is that the shape of the artificial spinal implant according to the invention can easily be adapted to the patient's anatomy during surgery.

The artificial spinal implant according to the invention comprises a thermoplastic elastomer comprising a hard phase and a soft phase.

The hard phase in the TPE comprises a rigid polymer phase with a melting temperature (Tm) or a glass transition temperature (Tg) higher than 35° C. The soft phase in the TPE comprises a flexible, amorphous polymer phase with a Tg lower than 35° C., preferably lower than 0° C. The Tm and Tg were determined on a dry sample.

The TPE, used according to the invention, comprises, for example, blends of the above-mentioned hard phase polymers with soft phase polymers and block copolymers. The hard and the soft phase can comprise one polymer type, but can also be composed of a mixture of two or more of the above-mentioned polymeric materials.

Preferably, the TPE, used according to the invention, is a block-copolymer. When the TPE is a block-copolymer, the TPE used in the artificial spinal implant comprises a thermoplastic elastomer comprising hard blocks and soft blocks, wherein the hard blocks comprise a polymer chosen from the group consisting of polyester, polyamide, polystyrene, polyacrylate and polyolefin and the soft blocks comprise a polymer chosen from the group consisting of polyether, polyester, polyacrylate, polyolefin and polysiloxane.

Examples of TPE block-copolymers according to the invention are block-copolyesterester, block-copolyetherester, block-copolycarbonateester, block-copolysiloxaneester, block-copolyesteramide, block-copolymer containing polybutylene terephthalate (PBT) hard blocks and poly(oxytetramethylene) soft blocks, block-copolymer containing polystyrene hard blocks and ethylene butadiene soft blocks (SEBS).

The hard blocks in the thermoplastic elastomer consist of a rigid polymer, as described above, with a Tm or Tg higher than 35° C. In principle the different polymers as described above can be used as the hard blocks. Here and in the rest of the description a polycarbonate is understood to be a polyester.

Also copolymers of esters, amides, styrenes, acrylates and olefins can be used as the hard polymer block as long as the Tm or Tg of the hard polymer block is higher than 35° C. Preferably, the hard block of the TPE is a polyester block.

More preferably, in the TPE comprising a hard polyester block, the hard block consists of repeating units derived from at least one alkylene glycol and at least one aromatic dicarboxylic acid or an ester thereof. The alkylene group generally contains 2-6 carbon atoms, preferably 2-4 carbon atoms. Preferable for use as the alkylene glycol are ethylene glycol, propylene glycol and in particular butylene glycol. Terephthalic acid, 2,6-naphthalenedicarboxylic acid and 4,4′-diphenyldicarboxylic acid are very suitable for use as the aromatic dicarboxylic acid. Combinations of these dicarboxylic acids, and/or other dicarboxylic acids such as isophthalic acid may also be used. Their effect is to influence the crystallization behavior, e.g. melting point, of the hard polyester blocks.

Most preferably, the hard block is polybutyleneterephthalate.

The soft blocks in the thermoplastic elastomer consist of a flexible polymer, as described above, with a Tg lower than 35° C. In principle the polymers as described above can be used as the soft blocks. Here and in the rest of the description a polycarbonate is understood to be a polyester.

Also copolymers of ethers, esters, acrylates, olefins and siloxanes can be used as the soft polymer block as long as the Tg of the soft polymer block is lower than 35° C.

Preferably, the soft block comprises a polyester or a polyether; more preferably an aliphatic polyester or polyether. A particular advantage of TPE's comprising polyester, or polyether soft blocks is that aliphatic polyesters, and polyethers feature a high chemical stability. Especially, alkylene carbonates and aliphatic polyesthers are preferred as the soft block, which result in thermoplastic elastomers with particularly low moisture sensitivity and favourable adhesive properties. Preferably, the soft blocks in the TPE are derived from at least one alkylene carbonate and optionally, a polyester made up of repeating units derived from an aliphatic diol and an aliphatic dicarboxylic acid.

The alkylene carbonate can be represented by the formula

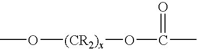

where
R=H, alkyl or aryl,
x=2-20.
   Preferably, R=H and x=6 and the alkylene carbonate is therefore hexamethylene carbonate.

The aliphatic diol units are preferably derived from an alkylenediol containing 2-20 C atoms, preferably 3-15 C atoms, in the chain and an alkylenedicarboxylic acid containing 2-20 C atoms, preferably 4-15 C atoms.

More preferably, the soft block comprises a polycarbonate.

It has been found that, with respect to the use in the artificial spinal implant according to the invention, in particular the thermoplastic block-copolyesters (TPC-ET, as defined in ISO 18064: 2003) have many advantages over other TPE's, such as the thermoplastic polyurethanes (TPU's) as described in for example U.S. Patent Application 2007/0050038A1, because of their improved mechanical properties, such as in particular low creep, low compression set, dimensional stability and resistance to moisture.

Most preferably, the TPE comprises a hard block comprising polybutyleneterephthalate and a soft block comprising polycarbonate. Optionally, this TPE is chain-extended with, for example, diisocyanate.

Examples and the preparation of block-copolyether esters are for example described in the Handbook of Thermoplastics, ed. O. Olabishi, Chapter 17, Marcel Dekker Inc., New York 1997, ISBN 0-8247-9797-3, Thermoplastic Elastomers, 2nd Ed., Chapter 8, Carl Hanser Verlag (1996), ISBN 1-56990-205-4, and the Encyclopedia of Polymer Science and Engineering, Vol. 12, pp. 75-117, and the references contained therein.

In another embodiment of the invention polyethylene oxide (PEO) or a combination of polyethylene oxide and polypropylene oxide (PEO-PPO-PEO) can be used as the soft block, which has a good biocompatibility and was found to result in osteoconductive (e.g. bone-bonding) surfaces capable of osteointegration. The PEO soft block can, for example, be combined with a PBT hard block.

The ratio of the soft and hard blocks in the TPE used in the artificial spinal implant according to the invention may generally vary within a wide range but is in particular chosen in view of the desired modulus of the TPE. The desired modulus will depend on the structure of the spinal implant and the functionality of the TPE in it. Generally, a higher soft block content results in higher flexibility and better toughness.

The TPE according to the invention may contain one or more additives such as stabilizers, anti-oxidants, colorants, fillers, binders, fibres, meshes, substances providing radiopacity, surface active agents, foaming agents, processing aids, plasticizers, biostatic/biocidal agents, and any other known agents which are described in Rubber World Magazine Blue Book, and in Gaether et al., Plastics Additives Handbook, (Hanser 1990). Suitable examples of fillers, e.g. radiopaque fillers and bone-mineral based fillers, and binders are described in U.S. Pat. No. 6,808,585B2 in columns 8-10 and in U.S. Pat. No. 7,044,972B2 in column 4, I. 30-43, which are herein incorporated as a reference.

Suitable commercially available TPE's include Arnitel® TPE (DSM Engineering Plastics), in particular Arnitel® E (polyether ester, PTMEG), Arnitel® C, (polycarbonate-ester, PHMC) and Arnitel® P (polyether ester, polyols, polypropylene and polyethylene). Particularly suitable Arnitel® grades include 55D, EL250, EM400, EM450, EM550, EM630, EL740, PL380, PL381, PM381, PL580, PM581, 3103, 3104, and 3107.

TPE's, in particular thermoplastic block copolyesters have been the subject of numerous FDA regulatory approvals. Specifically, Arnitel® copolyesters have been listed under the Drug Master Files 13260, 13261, 13263, 13264, 13259, and 13262. Additionally, these compositions have been cleared for permanent use in the human body (510(k) K990952, K896946). According to the FDA MAUDE database, adverse events dating back to prior April, 2000 are mild and due to mechanical failure (see catalogue number 8886441433, 447071, 8886471011V, and 8886470401). The absence of adverse effects due to material confirms the long-term biocompatibility of these compositions.

Arnitel® E grades are in compliance with the code of Federal regulation, issues by the Food and Drug Administration (FDA) 21 CFR 177.2600 (rubber articles for repeated use) in the USA, the so-called FDA approval. Moreover, US Pharmacopoeia approvals were received for the following Arnitel® grades: EM400, EM450, EM550, EM740, PL580 and 3104 (USP Class VI), and PL380 and PM381 (USP Class IV).

Moreover multiblock poly(aliphatic/aromatic ester) (PED) copolymers as described in M. El Fray and V. Altstädt, Polymer, 44 (2003) pp. 4643-4650 can suitably be used as the TPE according to the invention.

The spinal implant according to the invention can be produced in many different ways. Known techniques include (co-)injection molding, (co-)extrusion molding, blow molding or injection overmolding.

The temperature and other processing conditions at which the TPE can best be processed depends on the melting temperature, the viscosity and other rheological properties of the TPE and can easily be determined by the person skilled in the art once said properties are known. The above mentioned Arnitel® grades have melting temperatures (measured according to ISO 11357-1/-3) between 180 and 221° C. and are preferably processed at temperatures between 200 and 250° C.

The TPE's according to the invention, in particular Arnitel® TPE's, can be sterilized by any known means.

The TPE's according to the invention can be cut with a fluid jet for customizing the implant shape to the patient's anatomy. Such fluid jets are described in U.S. Pat. No. 6,960,182 and are commercially provided by Hydrocision, Inc. (Billerica, Mass.). The ability to customize an implant with a fluid jet represents a significant advance over the current standard of practice, where grinding tools (e.g. Dremel) are used to abrade the surfaces of implants, which result in damaged implant surfaces, possible introduction of wear particles in the operating room, etc. For instance, in F. W. Chan et al., “Is unidirectional wear testing appropriate for total disc replacement implants?” Global Symposium on Motion Preservation technology (SAS), New York, 2005, it is shown that surgeons abraded the posterior corner of a Maverick lumbar disc implant (Medtronic) using a Dremel tool in an effort to relieve nerve root compression. This would be much easier when using the TPE according to the invention and a fluid jet.

For spinal applications, implants are subjected to complex loading. This includes precompression in the axial direction and cyclic loads which represent a variety of physical activities. A spinal implant needs to be capable of absorbing shocks. For this type of applications hard-soft block systems are unique because they have a crystalline (hard block) component which is very resilient to mechanical forces. Moreover they are easily processible to provide a variety of designs and possess exceptional flex fatigue, which can be measured according to e.g. ISO 132 in which Arnitel® TPE has been demonstrated to survive an excess of 15 million cycles. This property is especially important for devices which undergo many flexural cycles, such as artificial disc replacements and dynamic stabilization devices.

In the past, thermoplastic as well as cross-linked polyurethane systems have been used. These have been subject to failure by creep, which decreases the height of the disc space over time (European Spine Journal (2007) (Suppl. 1): S13, EuroSpine 2007 conference industry workshop, TransS1, Inc.). Loss of disc height is one of the symptoms of degenerative disc disease and one of the key reasons for back pain and indications for spinal fusion, disc replacement, and other procedures. Crosslinked systems are not easily processed via traditional molding processes. This significantly restricts the range of designs which can be considered.

Alternatively, other non-elastomeric engineering plastics such as PEEK can be used. However, these are not elastomeric and are not capable of absorbing shock energy; this limits their effectiveness as a motion-preserving implant material. Spinal implants made of PEEK transfer shocks rather than absorbing them. For, example interspinous process spacers made from PEEK transfer shocks to adjacent spinous processes, rather than absorbing them, resulting in breakage of the spinous processes (European Spine Journal (2007) (Suppl. 1): S22). Disc and/or nucleus replacement designs made of such non-elastomeric engineering plastics (US2005/033437A1) require a pivot point in the implant to function. These do not reproduce human anatomy, which has the pivot point at the facet joint, rather than in the disc space. U.S. Pat. No. 6,973,678B2 attempts to provide for shock absorption with mechanical designs, however, these are still restricted to ball-and-socket type designs with a pivot point in the disc space.

Thermoplastic elastomers also provide the advantage of MRI compatibility over metals. In many discs metal components are applied, for example end plates in the Maverick and Charité designs of Medtronic and DePuy Spine, respectively, cause MRI and CAT scan artifacts. In contrast, polymer materials such as TPE are both MRI and CAT scan compatible (US2005/0033437A1).

The use of thermoplastic elastomers comprising hard and soft block polymers offers many advantages in disc design, for example for cervical disc and lumbar disc replacement implants. First of all, thermoplastic elastomers enable non-pivoting (e.g. non-ball-and-socket) designs, enabling designs which reproduce the spine's natural center of rotation, resulting in natural loading of facet joints while minimizing expulsion of the spinal implants.

In particular interspinous process spacers often comprise hard materials, e.g. piercing spinal ligaments, with soft materials, e.g. for elastic shock absorption & spacing). It is therefore important that such materials can be combined in one device. Arnitel® is known to feature good adhesion to for example other (harder of softer) grades of Arnitel® and metals.

An important requirement for all spinal implant devices is that they must withstand continuous compressive axial loading and flex modes (e.g. shock, cyclic, precompression, etc.) without creep.

The use of non-cross-linked TPE's according to the invention offers a possibility to reproduce both hard (end-plate) and soft (disc nucleus) as well as anisotropic properties of natural anatomy. Prior art, e.g. U.S. Patent Application 2007/0050038A1 is only capable of producing this in a cross-linked polyurethane system which restricts design to an (extruded) monolith.

Spinal implants comprising TPE's according to the invention can be produced in radiopaque versions for easy visualization of implant under X-ray. This can be accomplished by one skilled in the art of polymeric fillers and biocompatible materials. For example, barium sulfate, zirconium dioxide, hydroxyapatite, tricalcium phosphate, and other substances which impart radiopacity are described in U.S. Pat. No. 6,808,585 and U.S. Pat. No. 7,044,972 and incorporated here by reference.

Moreover it is possible to produce a fully MRI/CT-compatible implants by making them entirely of the TPE according to the invention. This is particularly important for certain classes of implants where subsequent diagnosis may be necessary. For example, interspinous process spacers (e.g. Kyphon Aperius, Abbott Spine Wallis, Medtronic DIAM) are intended to delay and/or prevent subsequent procedures (e.g. laminectomy, spinal fusion, or disc arthroplasty). Therefore, the ability to image soft tissues with MRI and/or CAT scans is advantageous in evaluating future therapeutic options.

As already mentioned above, a particular advantage of the use of a TPE according to the invention, in particular a block-copolyester, is its very good adhesion to different materials, for example to a different TPE, e.g. a TPE with a different stiffness or modulus, or a metal. This makes the material particularly suitable for application with for example a metal, for example Ti₆Al₄V, in overmolding. This property is expressed as a high peel strength. Preferably the peel strength is higher than 6 N/cm, measured according to ISO/IEC standard 7810.

In Biomaterials, 1992 13 (9), pp 585-593 it was demonstrated that the hydrolytic stability of block copolyester compositions clearly outperforms that of polyurethanes.

The use of TPE's according to the invention provides the ability to meet requirements without articulating surfaces, which minimizes the occurrence of wear, particles and/or reactions.

Examples of known artificial spinal implant designs that can be made partially or completely from the TPE according to the invention, or that can be partially or completely overmolded with the TPE according to the invention include artificial lumbar disc replacements, cervical disc replacements, implants for nucleus replacements, interspinous process spacers, and implants for dynamic stabilization. A more detailed overview and specific examples of said known artificial spinal implants, of which some are commercially available, is given below.

Many implants for lumbar disk replacement mentioned in Table 1 apply a so-called ball and socket design: hard articulating parts made of metal or hard polymer. The disadvantage of such designs is that they cannot absorb shock due to the non-elasticity of the used materials. Overmolding, e.g. by injection overmolding, the implant with the TPE according to the invention, completely or in part, will provide shock absorbing capacity to the implant.

Other designs, such as the CA Disc of Ranier Technology (Table 2), can be made completely of the TPE according to the invention. Multiple grades of TPE, preferrably Arnitel®, can be successively co-extruded and/or overmolded to produce an implant of varying modulus across the disc.

In contrast to the CA Disc technology, as described in US2007/043443A1, with TPE no post-processing crosslinking reaction is necessary. Furthermore, end plates of hard grades of TPE can be molded onto the implant core.

Compliant lumbar disc replacement implants, as listed in Table 2, typically include a core (U.S. Pat. No. 7,169,181, FIG. 1, 60; US2007/0043443A1, FIG. 4, 1; U.S. Pat. No. 7,153,325B2, FIG. 7, 76; US2005/0015150A1, FIG. 9, 400; and US2006/0259143A1, FIG. 3, 40). For these implant designs, the dynamic creep resistance and de Mattia flex fatigue resistance of Arnitel® TPE provide a distinct advantage for use as a core material. In some instances the core may be composed of an outer layer (annulus) generally of higher modulus (U.S. Pat. No. 5,171,281, FIG. 1, 4; and US2005/0015150A1 FIG. 9, 402) and a inner layer (nucleus) generally of a lower modulus polymer (U.S. Pat. No. 5,171,281, FIG. 1, 2; and US2005/0015150A1 FIG. 9 404). Here, the TPE provides a broad selection of properties for the two layers. Finally, the creep resistance and flex fatigue resistance of Arnitel® TPE provide an advantage over other elastomeric implant materials. Arnitel® TPE also provides sufficient adherence to metal endplates typically found in artificial lumbar disc implants (U.S. Pat. No. 7,169,181, FIG. 1, 20. 40; US2007/0043443A1, FIG. 4, 2; US2005/0015150A1, FIG. 9, 502, 504; and US2006/0259143A1, FIG. 3, 20, 30). As a final note, one could produce endplates from TPE or yet higher modulus polyester-based polymers. If all components of the implant were produced from polymer, the entire implant would enjoy the advantage of MRI compatibility versus traditional implants with metal endplates.

In Table 3 a list of known artificial spinal implants for cervical disc replacement is given.

TABLE 3
Implants for cervical disc replacement

| Company | Implant | Reference | Type |
|---|---|---|---|
| Biomet | Rescue | US2006/0085077A1 | Articulating |
| | | | Ball/Socket |
| Blackstone Medical | Advent | US2007/0073403A1, | Elastomer |
| | | WO2006/019370A1 | |
| Cervitech | PCM | US7267691B2 | Articulating |
| | | | Ball/Socket |
| Globus Medical | Secure-C | US20070055378A1 | Articulating |
| | | | Ball/Socket |
| LDR Spine | Mobidisc-C | US2005/0197706A1 | Articulating |
| | | | Ball/Socket |
| Medtronic Sofamor | Bryan | US7025787B2 | Elastomeric |
| Danek | | | |
| Medtronic Sofamor | Prestige | US6540785B1 | Articulating |
| Danek | | | Ball/Socket |
| NuVasive | Neo-Disc | US2008/0015697A1 | Elastomeric |
| Spinal Kinetics | M6-C | US2007/0050032A1 | Elastomeric |
| Stryker | Cervicore | — | Articulating |
| | | | Ball/Socket |
| Synthes | ProDisc-C | — | Articulating |
| | | | Ball/Socket |

Some of the known implants for cervical disk replacement, e.g. Medtronic's Bryan, Blackstone's Advent, NuVasive's Neo-Disc and Spinal Kinetics M6 already comprise a soft part, usually made of an elastomer. This part can be made of the TPE according to the invention resulting in a device with improved creep resistance and compression set (U.S. Pat. No. 7,025,787B2, FIG. 4, 60; US2007/0073403A1, FIG. 1, 104, FIG. 3, 304, FIG. 8, 804; US2008/0015697A1, FIG. 4, 40; US2007/0050032A1, FIG. 3, 130). As with lumbar disc replacement implants, endplates for cervical disc replacement implants could be produced from TPE or other polymers which are moldable with TPE to produce a fully polymer MRI-compatible implant (U.S. Pat. No. 7,025,787B2, FIG. 6, 20, 40; and US2007/0073403A1, FIG. 1, 402A, 402B, FIG. 3, 102A, 102B, FIG. 8, 802A, 802B). For complete MRI compatibility with an elastic yet creep-resistant jacket (US2008/0015697A1, FIGS. 9a, 9b, 9c, 118, 120) could incorporate TPE. In Table 4 a list of known artificial spinal implants for spinous process spacers is given.

TABLE 4
Implants for spinous process

| Company | Implant | Reference |
|---|---|---|
| Abbott Spine | Wallis | US6946000B2 |
| Biomet | ISS | US2006/0015181A1 |
| Globus | Flexus | US2006/0293662A1 |
| Kyphon | Aperius | WO2006/089085A2 |
| Medtronic Sofamor Danek | DIAM | US6626944B1 |
| Medtronic Sofamor Danek | TBA | US2005/0261768A1 |
| Paradigm Spine | CoFlex | U.S. Pat. No. 5,645,599 |
| Privelop | Spinos | EP1845876 |
| St. Francis Medical Technologies | X-Stop | US2005/0075634A1 |
| Zimmer Spine | TBA | US2007/0055373A1 |

The Wallis design of Abbott Spine (U.S. Pat. No. 696,400B2) could use TPE to form a creep-resistant, shock-absorbing and damping “wedge” (FIG. 1, 10). In addition, the band (FIG. 1, 54) could be produced from TPE to produce a band with elastic properties; this would improve take-up in band slack to reduce the risk of implant migration during extension of the spine.

The ISS design of Biomet (US2006/0015181A1) could potentially benefit from the high flex-fatigue of TPE (FIG. 12, 11) as well as the ability to combine with higher modulus TPE and/or other polymers (12, 13, 16, 17).

The Flexus design of Globus (US2006/0293662A1) would benefit from the creep-resistance and shock-absorbing properties of TPE where the spinous processes contact the implant (FIG. 37, 422).

A combination of both harder and softer TPE's can be used to provide both tissue-piercing and implant retention capability (for 420 and 423, respectively) and shock-absorbing capacity (for 422).

The interspinous portion of the DIAM design of Medtronic Sofamor Danek (U.S. Pat. No. 6,626,944B1, FIG. 1, 5) could be produced from TPE according to the invention. This would provide improved flex and compression fatigue combined with shock absorbing capabilities between the spinous processes. Likewise, the cord (FIG. 1, 8) could also be produced from TPE to yield a compliant and elastic yet creep-resistant cord.

The CoFlex design of Paradigm Spine (U.S. Pat. No. 5,645,599) could be substantially produced from TPE according to the invention to provide improved shock absorption and implant flexibility.

The Spinos design of Privelop (EP1845876) can be made entirely or partly of the TPE according to the invention or can be overmolded. For example, parts 2A and 2B can be either produced from TPE or overmolded on a metal substrate.

The X-Stop design of St. Francis Medical Technologies (US2005/0075634A1) can be made entirely or partly of the TPE according to the invention or can be overmolded. For example, 150 could be produced from and/or overmolded with a low-modulus, shock-absorbing TPE grade while the remaining parts, including but not limited to 111, 110, 132, and 104, could be produced from a higher-modulus TPE grade to provide tissue-piercing capacity as well as long-term fixing of the implant.

In the design of Zimmer (US2007/0055373A1) particularly part 19 can be made of or overmolded with the TPE according to the invention.

In Table 5 a list of known artificial spinal implants for dynamic stabilization is given.

TABLE 5
Implants for dynamic stabilization

| Company | Implant | Reference |
|---|---|---|
| Applied Spinal Technology | Stabilimax | US2005/0171543A1 |
| Globus Medical | AccuFlex | US6989011B2 |
| Impliant | TOPS | US7011685B2 |
| Innovative Spinal Technologies | Axient | US2006/0036240A1 |
| (IST) | | |
| Interventional Spine (formerly | Percudyne, | US2006/0122609A1 |
| Triage Medical) | PERPOS | U.S. Pat. No. 6,685,706 |
| LifeSpine | Prodigy | US2007/0118122A1 |
| Medicrea | Granvia | FR2867375 |
| N Spine | Nflex | US2005/0203517A1 |
| Scient'x | Isobar TTL | US6241730B1 |
| SpineVision | FlexPLUS | US2007/0129729A1 |
| X-Spine | — | US2008/0027549A1 |
| Zimmer | DTO | WO2008/115622A1 |
| Zimmer | Dynesys | EP0669109 |

Generally, devices for dynamic stabilization comprise one or more rod-shaped or rectangular shaped members connecting a number or screws for fixing the device to the spinal column. The rods and rectangular shapes in these devices are meant to be flexible in order to provide dynamic stabilization in contrast with traditional metal rods. Therefore one or more of these rods or rectangular members can be made of the TPE according to the invention to provide improved shock absorption.

Typically the “rods” are modified to allow some level of motion preservation instead of promoting spinal fusion. One example for this approach is to provide a mechanical spring (US2005/0171543A1, FIG. 4: 30, 32, FIG. 8: 212, 214; US2006/0036240A1, FIG. 4C, 44, FIG. 7, 74) or flexible elastomer element in a rod system (US2007/0118122A1, FIG. 5, 120, 121; US2005/0203517A1, FIG. 54, 287, 290; U.S. Pat. No. 6,241,730B1, FIG. 1, 7A; US2007/0129729A1, FIG. 1, 3; FIG. 2, 2, 4; US2008/0027549A1, FIG. 6, 64, FIG. 10, 64, 66; WO2008/115622A1, FIG. 1, 24, 36; EP0669109, FIG. 6, 1, 10). Alternatively, a large elastomer element can be used in place of elastomer elements in a rod-like system (U.S. Pat. No. 7,011,685B2, FIG. 7A, 64) or a hinge-like construction may be used (US2007/0118122A1, FIG. 1, 110) The advantages of using TPE over other elastomers in these designs include dynamic creep under tension and compression, improved crack growth resistance (as evidenced by ISO 132 “De Mattia” testing), and ease of melt processing TPE. The advantages of TPE over mechanical spring systems include fewer moving parts, less wear debris, higher reliability, and simplified production, assembly, and quality assurance.

Another approach is to provide a rod of modified stiffness (U.S. Pat. No. 6,989,011B2, FIG. 21, 200, FIG. 23, 260).

Other uses particularly suited for TPE include sheaths and sleeves for mechanical spring assemblies in dynamic stabilization systems (US2005/0171543A1, FIG. 9, 300; US2006/0036240A1, FIG. 7, 77; US2007/0118122A1, FIG. 1, 108, FIG. 13, 224, FIG. 17, 270) as well as screw-like implants (US2006/0122609A1, FIG. 3, 28). Here, the creep and flex fatigue resistance of TPE under many cycles of compression and tension is preferred over alternate materials.

In Table 6 a list of known artificial spinal implants for nucleus replacement is given.

TABLE 6
Implants for nucleus replacement

| Company | Implant | Reference |
|---|---|---|
| Biomet | Regain | WO03/065929A2 |
| CryoLife | BioDisc | US2007/0093902A1 |
| Disc Dynamics | DASCOR | US7001431B2 |
| Medtronic | — | US2007/0239279A1 |
| NuVasive (Pearsalls) | NeoDisc | WO2005/092248A1 |
| Pioneer Surgical | NUBAC | US2005/0033437A1 |
| Raymedica | Hydraflex | US2006/0237877A1 |
| Raymedica | PDN | U.S. Pat. No. 5,674,295 |
| Replication Medical | NeuDisc | US2005/0171611A1, |
| | | WO2007/095121A2 |
| Synthes | Gelifex SP and IP | WO2004/098756A2 |
| Synthes | — | US2005/0090901A1 |
| TransS1 | PNR | US2005/0113919A1 |
| Zimmer | Newcleus | U.S. Pat. No. 5,919,235 |

Several design types exist for nucleus replacements, all of which could benefit from the use of TPE's. Specifically, many nucleus replacements seek to directly replace the nucleus with a solid implant (WO03/065929A2, FIG. 15, 500; US2007/0239279A1, FIG. 1, 104A, 1048, 109; WO2005/092248A1 FIG. 1, 22; US2006/237877A1, FIG. 1, 22; U.S. Pat. No. 5,674,295 FIG. 1, 12, US2005/171611A1, FIG. 1, 21, 23, 25; U.S. Pat. No. 5,919,235). These could either be wholly or partially substituted by TPE, especially softer grades alone or in combination with hydrogels to produce a soft core of the nucleus replacement. In these instances, a TPE would substitute many of the load-bearing components of the nucleus replacement. The advantages afforded are improved crack growth resistance and especially dynamic creep resistance, which allow the implant to maintain its shape, flexibility and function over time In other cases, a woven TPE jacket or cover (US2006/237877A1, FIG. 1, 24; U.S. Pat. No. 5,674,295 FIG. 1, 14; WO2005/092248A1, FIG. 1, 7; US2005/171611A1, FIG. 1, 3; WO2007/095121A2 FIG. 1, 14) can be produced from TPE. Here the advantage is an elastic jacket material which can expand as the hydrogel at the core of the nucleus replacement expands, while still maintaining shape over time (dynamic creep resistance). Alternative designs for disc nucleus replacements include injectible nucleus replacements. In these designs, a woven fabric, membrane, or other type of structure may be used to contain in injected, curable or other filler materials (US2007/093902A1, FIG. 1, 10-2; U.S. Pat. No. 7,001,431B2, FIG. 4, 34, 38; US2005/090901A1, FIG. 1, 1; and US2005/0113919A1, FIG. 5, 18). TPE's could effectively either produce these woven or membrane containment. Here the dynamic creep resistance plays a critical role after implantation and expansion of the device. Finally, US2005/033437A1 describes a nucleus replacement which is very similar to a lumbar or cervical disc replacement and could incorporate TPE's as such, described earlier in this description.

The invention also relates to the use of TPE's in spinal implants, in particular for lumbar disk replacement, cervical disk replacement, nucleus replacement, dynamic stabilization or as interspinous process spacer.

The invention also relates to the use of the artificial spinal implants according to the invention in procedures for motion preservation in the spine, for example dynamic stabilization, disc and/or nucleus replacement, annulus repair, facet joint repair, kyphoplasty, vertebroplasty, laminectomy, and spinal stenosis treatment.

### EXAMPLES

Arnitel® grades have melting temperatures (measured according to ISO 11357-1/-3) between 180 and 221° C. and were processed at temperatures between 200 and 250° C. The samples were injection molded.

The test samples were stored at room temperature for at least 10 days before conducting the experiments.

Moduli were determined according to ISO 527; sample type 5A.

### Materials

Arnitel® 55D (hard block: polybutylene terepthalate (PBT), soft-block: polycarbonate, modulus 140 MPa) from DSM N.V.
Arnitel® EL250 (hard block polybutylene terepthalate (PBT), soft-block: polytetramethyleneoxide (PTMO), modulus 25 MPa) from DSM N.V.
Arnitel® EM400 (hard block: polybutylene terepthalate (PBT), soft-block:

polytetramethyleneoxide (PTMO), modulus 50 MPa) from DSM N.V.
Arnitel EM 460 (hard block polybutylene terepthalate (PBT), soft-block: polytetramethyleneoxide (PTMO), modulus 100 MPa) from DSM N.V.
Arnitel® EM550 (hard block polybutylene terepthalate (PBT), soft-block: polytetramethyleneoxide (PTMO), modulus 200 MPa) from DSM N.V.
Arnitel® EM630 and 630-H (hard block polybutylene terepthalate (PBT), soft-block: polytetramethyleneoxide (PTMO), modulus 310 MPa) from DSM N.V. (H means heat stabilized)
Arnitel® EM740 (hard block polybutylene terepthalate (PBT), soft-block: polytetramethyleneoxide (PTMO), modulus 1100 MPa) from DSM N.V.
Arnitel® PL380 (hard block polybutylene terepthalate (PBT), soft block: Polyethyleneoxide-polypropyleneoxide-polyethyleneoxide (PEO-PPO-PEO), modulus 60 MPa from DSM N.V.
Elastollan® 1190A TPU; a polyether-urethane from BASF A.G.
Elastollan® 1195A TPU; a polyether-urethane from BASF A.G.

### Example I Axial Stiffness of an Artificial Lumbar Disc

To investigate whether Arnitel® can meet the axial stiffness requirements for a lumbar disc, calculations were performed for Arnitel® EL250 and EM400. The required axial stiffness is between 1000 and 3500 N/mm (U.S. Pat. No. 5,171,281). For a solid circular disc with diameter (D)=30 mm and height (H)=10 mm the axial stiffness k under uniaxial compression can be calculated with the formula k=EA/H (k=axial stiffness, E=modulus, A=cross-sectional area=πD²/4, H=height):
EL250: E=25 MPa→k=1800 N/mm
EM400: E=50 MPa→k=3500 N/mm

The modulus E was determined according to ISO 527.

Based on these calculations it was concluded that in particular the softer Arnitel® grades possess the required axial stiffness when used as such.

### Example II Compression Stress Upon Axial Load on Lumbar Disc

To investigate whether Arnitel® can withstand the compressive stress experienced when loaded as a lumbar disc a calculation was performed for Arnitel® EM400. A typical axial load is F=400 N (LeHuec et al., J. Spine Dis. & Tech. 16 (4) 346-351; O'Leary et al., The Spine J. 2005, 590-599; Grauer et al, The Spine J., 2006, 6, 659-666). Stress could be calculated from the axial load F and the cross-sectional area A (πD²/4, D=diameter) with the formula σ=F/A. For a disc of D=30 mm and a load F=400 N the compressive stress is 0.57 MPa. This value is about one order of magnitude below the yield strength of Arnitel® EM400, so it can safely be assumed that this material is able to with stand the stress levels encountered in the application.

### Example III Torsion of a Lumbar Disc

Assuming torsion over a maximum angle of α=2.5° for a circular disc of diameter D=30 mm, R=15 mm, and H=10 mm.

The maximum shear strain at the side is γₘₐₓ=Rα/H=15*2.5*(π/180)/10=0.052. For Arnitel® EM400 with E=50 MPa, determined according to ISO 527, and assuming Poisson's ratio ν=0.4, the shear modulus G=E/2(1+ν)=50/2(1.4)=17.9 MPa.

Maximum shear stress τₘₐₓ=G×γₘₐₓ=0.93 Nm.

Torque=½*π*τₘₐₓ*R³˜5 Nm which (for α=2.5°) is 2 Nm/°.

According to Parsons et al, U.S. Pat. No. 5,171,281 torques are between 0.8 and 3 Nm/°. The torsion stiffness of an Arnitel® EM 400 circular disc was thus within the desired range.

### Example IV Comparison of Tensile and Creep Properties

The tensile modulus and the creep properties were determined at room temperature according to ISO 527. The sample used was type 5A.

| | Tensile | Tensile Modulus in |
|---|---|---|
| Material | Modulus | strain domain 0-5% |
| Arnitel ® PL380 | 60 MPa | 41 MPa |
| Arnitel ® EM400 | 50 MPa | 38 MPa |
| Elastollan ® 1195A | 65 MPa | 47 MPa |

The determined tensile modulus of three materials was similar.

Stress level: 2.5 MPa

| | Initial | Strain after |
|---|---|---|
| Material | strain | 1 hour |
| Arnitel ® PL380 | 5.8% | 7.4% |
| Arnitel ® EM400 | 6.9% | 9.0% |
| Elastollan ® 1195A | 5.5% | 10.5% |

Stress level: 5.0 MPa

| | Initial | Strain after |
|---|---|---|
| Material | strain | 1 hour |
| Arnitel ® PL380 | 20.4% | 35.1% |
| Arnitel ® EM400 | 27.1% | 51.2% |
| Elastollan ® 1195A | 17.7% | 56.8% |

The tables show that the two TPE materials, having a similar tensile modulus as the Elastollan® material, were clearly more creep resistant than the comparative Elastollan® 1195A material.

This means that in the artificial implants fabricated using Arnitel® PL380 and EM400 are less susceptible to creep failure.

### Example V Comparison of Dynamic Creep Properties

### Experiment

Cylindrical samples having a 13 mm diameter and 6 mm height were mounted between the plates of a MTS 810-II servo-hydraulic tensile tester. The samples were loaded force controlled by a harmonically time varying compressive force. The cycle frequency of the force signal was 0.25 Hz. The maximum compressive stress during a cycle was 4 MPa whereas the minimum compressive stress was 0.4 MPa. The experiments were carried out in an oven at 37° C. The stress levels that were applied were derived from ASTM 2423-05, and were chosen to be higher by a factor 4.

### Results

The sample compression at the maximum and minimum stress during a cycle was monitored as a function of cycle number. The results are summarized in the tables below.

Arnitel ® EM400

| | Compressive strain | Compressive strain |
|---|---|---|
| Cycle number | at 4 MPa | at 0.4 MPa |
| 1 | 4.4% | 1.4% |
| 20000 | 4.7% | 1.9% |

Elastollan ® 1190A

| | Compressive strain | Compressive strain |
|---|---|---|
| Cycle number | at 4 MPa | at 0.4 MPa |
| 1 | 7.8% | 2.0% |
| 20000 | 11% | 6.1% |

By comparing the compressive strain at the minimum stress level it was observed that the Arnitel® EM400 material clearly showed more creep resistant behavior than the Elastollan® 1190A material in the tests. For the Arnitel® EM400 material the compressive strain had increased from 1.4% to 1.9% over 20000 cycles, which is a relative increase of about 35%, whereas for the Elastollan® 1190A material the compressive strain had increased from 2.0% to 6.1% corresponding to a relative increase of more than 200%.

### Example VI Biocompatibility

Samples of Arnitel® EL250, EM400, and EM740 were tested under GLP conditions according to ISO 10993 parts 3, 5, 6, 7, 10, and 11:
ISO 10993-3 Tests for genotoxicity, carcinogenicity, and reproductive toxicity.
ISO 10993-5 Tests for in vitro cytotoxicity.
ISO 10993-6 Test for local effects after implantation.
ISO 10993-7 Ethylene oxide residuals.
ISO 10993-10 Test for irritation and delayed-type hypersensitivity.
ISO 10993-11 Test for systemic toxicity

Each of these material grades passed all of the above biocompatibility tests, demonstrating the safety of Arnitel® TPE as an implant material.

### Example VII Sterilization Testing

Samples of Arnitel® types CM551, EL250, EM550, EM630, EM630-H, EM400, EM460, EL630, and EL740 were tested for the effects of gamma sterilization up to 100 KGray (roughly 4 times a typical sterilization dose). These samples were subsequently mechanically tested to determine the effects on E-modulus, Stress at Break, and Strain at Break. In all instances little or no changes in the material properties were observed.

### Example VIII Flex Fatigue Testing

Arnitel® EM400 and Elastollan® 1190A TPU were tested according to the ISO 132 deMattia test. The results showed favorable crack growth numbers for Arnitel® EM400.

| | Arnitel ® EM400 crack | Elastollan ® 1190A crack |
|---|---|---|
| # cycles | length (mm) | length (mm) |
| 0 | 2.0 | 2.0 |
| 19000 | 6.6 | 6.9 |

1. An artificial spinal implant comprising a thermoplastic elastomer comprising a hard phase and soft phase, wherein the hard phase comprises a polymer chosen from the group consisting of polyester, polyamide, polystyrene, polyacrylate and polyolefin and the soft phase comprises a polymer chosen from the group consisting of polyether, polyester, polyacrylate, polyolefin and polysiloxane.

2. An artificial spinal implant according to claim 1, where in the hard phase and the soft phase are present in a block copolymer, wherein the hard blocks are chosen from the group consisting of polyester, polyamide, polystyrene, polyacrylate and polyolefin and the soft blocks are chosen from the group consisting of polyether, polyester, polyacrylate, polyolefin and polysiloxane.

3. The artificial spinal implant according to claim 2, wherein the hard block is a polyester hard block.

4. The artificial spinal implant according to claim 3, wherein the polyester hard block consists of repeating units derived from at least one alkylene glycol and at least one aromatic dicarboxylic acid or an ester thereof.

5. The artificial spinal implant according to claim 4, wherein the polyester hard block is polybutyleneterephthalate.

6. The artificial spinal implant according to claim 2, wherein the soft block comprises an aliphatic polyester or polyether.

7. The artificial spinal implant according to claim 6, wherein the soft block comprises polycarbonate.

8. The artificial spinal implant according to claim 2, wherein the hard block is polybutyleneterephthalate and the soft block comprises polycarbonate,

9. Use of a thermoplastic elastomer comprising a hard phase and soft phase, wherein the hard phase comprises a polymer chosen from the group consisting of polyester, polyamide, polystyrene, polyacrylate and polyolefin and the soft phase comprises a polymer chosen from the group consisting of polyether, polyester, polyacrylate, polyolefin and polysiloxane in a spinal implant.

10. Use of a thermoplastic elastomer comprising a hard phase and soft phase, wherein the hard phase comprises a polymer chosen from the group consisting of polyester, polyamide, polystyrene, polyacrylate and polyolefin and the soft phase comprises a polymer chosen from the group consisting of polyether, polyester, polyacrylate, polyolefin and polysiloxane in procedures for motion preservation in the spine, for example dynamic stabilization, disc and/or nucleus replacement, annulus repair, facet joint repair, kyphoplasty, vertebroplasty, laminectomy, and spinal stenosis treatment.

11. The artificial spinal implant according to claim 1, or the use according to claim 9 or 10, wherein the spinal implant is applied for lumbar disc replacement, cervical disc replacement, nucleus replacement, dynamic stabilization or as spinous process spacer.

12. Use of the artificial spinal implant according to claim 1, in procedures for motion preservation in the spine, for example dynamic stabilization, disc and/or nucleus replacement, annulus repair, facet joint repair, kyphoplasty, vertebroplasty, laminectomy, and spinal stenosis treatment.
